(19) United States
(12) Patent Application Publication
(10) Pub. No.: US 2018/0192913 A1
(43) Pub. Date: Jul. 12, 2018

(54) IDENTIFICATION OF DYNAMIC HYPERINFLATION USING A COMBINATION OF EXPIRATORY FLOW AND RESPIRATORY CARBON DIOXIDE SIGNALS

(71) Applicant: Mermaid Care A/S, Nørresundby (DK)

(72) Inventors: Stephen Edward Rees, Gistrup (DK); Sebastian Larraza Rico, Aalborg (DK); Dan Stieper Karbing, Aalborg (DK)

(21) Appl. No.: 15/739,573

(22) PCT Filed: Jun. 28, 2016
(86) PCT No.: PCT/DK2016/050224

§ 371 Date: Dec. 22, 2017

(30) Foreign Application Priority Data
Jun. 30, 2015 (DK) PA 2015 70420

(51) Int. Cl. A61B 5/083; A61B 5/087; A61B 5/00; A61M 16/00

(52) U.S. Cl. CPC A61B 5/0836; A61B 5/087; A61B 5/7275; A61M 16/0051; A61M 2205/502; A61M 2016/0042; A61M 2205/18; A61M 2230/432; A61M 16/024

(57) ABSTRACT

A method is provided for identification of an increase in the gas volume of the lung caused by the inability of a patient to expire completely, known as dynamic hyperinflation or gas trapping.

### FIELD OF THE INVENTION

The present invention relates to a method for identification of an increase in the gas volume of the lung caused by the inability of a patient to expire completely, known as dynamic hyperinflation or gas trapping. The method applies analysis of a patient's pattern of expiratory flow, along with the pattern of respiratory, e.g. expiratory, carbon dioxide level, for example the capnography signal. Analysis of these signals enables identification of dynamic hyperventilation, along with identification of change in the degree of dynamic hyperinflation indicating improvement or worsening in the patient state.

### BACKGROUND OF THE INVENTION

Patients with severe acute illness, often requiring mechanical support for ventilation, usually reside in the intensive care unit (ICU) of the hospital. Here they are monitored intensively using a range of machines, which measure physiological variables related to pulmonary function. These machines include the mechanical ventilator, from which flow and pressure measurements can be made, and capnometers, which allow measurement of the partial pressure of carbon dioxide in the respiratory gas.

Mechanical ventilation is an essential technology in many patients. However, therapy with mechanical ventilation includes potential risk to the patient. Excessive pressures or volumes can damage the lung (1; i.e. Reference ‘1’ given below). Poor setting of the ventilator, or particular breathing patterns, can result in a reduced time for expiration, meaning that the patient cannot expire the complete volume inhaled during inspiration (2,3,4). This can lead to over inflation of the lungs, resulting in higher lung volume and pressure, which, in turn, can result in lung damage or compromise blood circulation in the lung (2,3). The increase in volume due to this cause is known as dynamic hyperinflation or gas trapping, the increase in pressure due to this cause is known as autoPEEP (auto positive end expiratory pressure) or intrinsic PEEP (2,3,4).

Identification of dynamic hyperinflation or autoPEEP is not simple. Pauses in mechanical ventilation at the end of expiration have been used to extend the expiratory time, with identification of significant flow or pressure changes during this period providing a clear identification of these effects (2,3,4). An end expiratory hold has also been used, where following end expiratory respiratory tube occlusion the pressure measured in the respiratory tube equilibrates with that in the lungs and therefore describes autoPEEP (2,3,4). Analysis of expiratory flow profile has also been used (5,6,7). Characterisation of the flow profile using time constants has been performed to identify those with an underlying physiological problem in expiratory flow (7). In addition, analysis of the flow signal not returning to zero at the end of expiration has been used to indicate the possible presence of dynamic hyperinflation or auto PEEP (5,6).

All of these measurements suffer from the problem that they can usually only be applied in completely paralysed, highly sedated patients who have little or no spontaneous breathing effort (3,6). The majority of mechanically ventilated patients residing in the ICU have spontaneous breathing effort. In these patients, prolonging expiration or occluding the respiratory tube can cause the patient to fight for breath, resulting in both discomfort and a corrupted measurement of dynamic hyperinflation and autoPEEP. Analysis of the expiratory flow signal is also difficult as breathing varies greatly on a breath by breath basis in these patients (3). Expiratory flow profiles can vary greatly, meaning that it is not possible to estimate time constants, which consistently describe the patients flow pattern. In addition, in some breaths the flow profile may return to zero which in others full expiration may occur, meaning that interpretation of the patient state is difficult (3). For these patients measurement of dynamic hyperinflation or auto-PEEP is usually only performed with esophageal pressure measurements (3), which are not routinely performed in the ICU.

Hence, an improved method to identify dynamic hyperinflation would be advantageous, and in particular a method which exploits the breath by breath variability in expiration seen in patients with spontaneous breathing effort.

### OBJECT OF THE INVENTION

It is a further object of the present invention to provide an alternative to the prior art. In particular, it may be seen as an object of the present invention to provide a method that solves the above mentioned problems of the prior art with identification of dynamic hyperinflation in patients residing in intensive care units (ICUs).

### SUMMARY OF THE INVENTION

The prior art strategies for detection of dynamic hyperinflation, based upon prolonged expiration, expiratory occlusion or analysis of the expiratory flow signal may have drawbacks.

Firstly, prolonged expiration prevents the patient initiating a new inspiration. In the case where the patient tries to initiate a new inspiration any residual volume trapped in the lungs will not be expired but remain in the lungs due to the patients inspiratory effort.

Secondly, occlusion of the respiratory tube at the end of expiration prevents the patient initiating a new inspiration. In this case, the patient will experience the discomfort of inspiring against a closed respiratory circuit. The negative pressure generated by the patient effort will seriously corrupt the pressure reading used to determine the autoPEEP level.

Third, estimation of expiratory flow profiles using calculation of the time constant(s) of expiratory flow is seriously limited in patients with spontaneous breathing effort. These patients often have irregular breathing, meaning that the profile of expiratory flow can change dramatically between breaths. This can be further compounded if patients' expiration is not completely passive, meaning that respiratory muscles might be used to force expiration.

Fourth, assessment of the dynamic hyperinflation from inspection of the inspiratory flow profiles' return to zero may also be limited in patients with spontaneous breathing. The irregular pattern of expiration seen in these patients may mean that interpretation of the degree of dynamic hyperinflation may vary substantially depending upon the breath(s) used for analysis.

Thus, the above-described object and several other objects are intended to be obtained in a first aspect of the invention by providing a computer-implemented method for determining the risk of a subject of having dynamic hyperinflation and/or increased auto positive end expiratory pressure (autoPEEP) in a subject, the method comprising
a) providing a first input, indicative of the expiratory flow of the subject;
   and
b) providing second input, indicative of the carbon dioxide level in expired gas of the subject; and
c) comparing said first and second input to one or more corresponding reference levels; and
d) if said first and second input deviate significantly from said one or more reference levels, said subject is at risk of having dynamic hyperinflation and/or increased autoPEEP; or if said first and second input do not deviate significantly from said one or more reference levels, said subject is not at risk of having dynamic hyperinflation and/or increased autoPEEP.

In a second aspect, there is provided a computer-implemented method for determining a change in dynamic hyperinflation and/or auto positive end expiratory pressure (autoPEEP) in a subject, the method comprising
a) providing a first input indicative of the expiratory flow profile of the subject;
   and
b) providing a second input indicative of the carbon dioxide level in expired gas of the subject; and
c) comparing said first and second input to corresponding input provided from said subject from a previous point in time; and
d) if said first and second input deviate significantly from said one or more corresponding input provided from said subject from a previous point in time, said subject is at risk of having dynamic hyperinflation and/or increased autoPEEP; or if said first and second input do not deviate significantly from said one or more corresponding input provided from said subject from a previous point in time, said subject is not at risk of having dynamic hyperinflation and/or increased autoPEEP.

Thus, the method of the invention provides an indication as to the degree of dynamic hyperinflation (first aspect), and as to changes in dynamic hyperinflation relevant to clinical practice over time (second aspect), which may allow a clinician to take appropriate action, such as modifying the settings of the mechanical ventilator, for example the level of pressure support, positive end exipiratory ratio; or in controlled ventilator modes, the inspiratory:expiratory ratio.

The present invention is particularly—but not exclusive—advantageous, in that measurement and analysis, i.e. comparison, of flow data and carbon dioxide data is applied to obtain hitherto unavailable insight about the dynamic hyperinflation and the changes of hyperinflation for a patient. More specifically, it will be demonstrated below that valuable insights about a patient state and change of patient state, cf. FIGS. 4 and 5, respectively, with respect to hyperinflation may be obtained with the present invention. Insights that—to the best knowledge of the inventors—was previously not available in this field.

In the broadest sense, the invention may be advantageously applied to assist in estimating the risk of dynamic hyperinflation and/or increased autoPEEP in two-result fashion, i.e. ‘at risk’ or ‘no risk’, but the invention may of course also output more nuanced level of this risk, both qualitatively and in a quantified manner. Thus, in a quantitative manner it could be a number, such as a percentage, indicating the risk, and if the risk is given in a qualitative manner it could be e.g. a three-level risk regime, e.g. ‘no risk’, ‘little risk’ and ‘high risk’, a four-level risk regime, and so forth.

Risks may be outputted and indicated to a user, e.g. a clinician, in any kind of suitable graphical user interface (GUI), by sounds/alarms, or other human-machine interfaces, and/or stored for later use, e.g. analysis and assessment by a clinician.

It may be mentioned that—in the context of the present invention—the said indications are intended for assisting or guiding e.g. clinician in making decisions of a therapeutic and/or a diagnostic character. Thus, the present invention is not designed to perform an actual diagnosis, merely providing intelligent information, i.e. indications that may assist the clinician in performing the subsequent step of making the intellectual exercise of providing a diagnosis of the patient state, the diagnosis may then be followed be an action of therapeutic character, if needed. Thus, the present invention does not necessarily include the intellectual step strict sensu of making a diagnosis but rather assists in providing information beneficial for making an actual diagnosis. In one aspect, the invention may be implemented as a decision support system (DSS) for medical personnel involved in monitoring of the subject, performing medical assessment of the subject and/or planning of medical treatment of the subject, e.g. the patient. The step of providing first and/or second input need not be performed by interaction(s) with the subject, but could be implemented by receiving such corresponding first and/or second input(s), i.e. not performing the actual measurement(s) on the subject.

In a third aspect, there is provided a computer-implemented method for determining the risk of a subject of having dynamic hyperinflation and/or increased auto positive end expiratory pressure (autoPEEP) in a subject, the method comprising
a) providing a first input, indicative of the expiratory flow of the subject;
   and/or
b) providing second input, indicative of the carbon dioxide level in expired gas of the subject; and
c) comparing said first and/or second input to one or more corresponding reference levels; and
d) if said first and/or second input deviates significantly from said one or more reference levels, said subject is at risk of having dynamic hyperinflation and/or increased autoPEEP; or if said first and/or second input do not deviate significantly from said one or more reference levels, said subject is not at risk of having dynamic hyperinflation and/or increased autoPEEP.

In a fourth aspect, a computer-implemented method for determining a change in dynamic hyperinflation is provided and/or auto positive end expiratory pressure (autoPEEP) in a subject, the method comprising
a) providing a first input indicative of the expiratory flow profile of the subject;
   and/or
b) providing a second input indicative of the carbon dioxide level in expired gas of the subject; and
c) comparing said first and/or second input to corresponding input provided from said subject from a previous point in time; and
d) if said first and/or second input deviate significantly from said one or more corresponding input provided from said subject from a previous point in time, said subject is at risk of having dynamic hyperinflation and/or increased autoPEEP; or if said first and/or second input do not deviate significantly from said one or more corresponding input provided from said subject from a previous point in time, said subject is not at risk of having dynamic hyperinflation and/or increased autoPEEP.

The first input may be integrated over time. Thus, in an embodiment, the first input indicative of the expiratory flow of the subject is further integrated over time for an individual expiratory breath of the subject, and if the integrated area is below a certain threshold area then the specific expiratory breath is categorised as incomplete, and/or if the integrated area is above a certain threshold area then the specific expiratory breath is categorised as complete.

In another embodiment, a value of the time derivative of the first input at the end of the said specific expiratory breath is additionally applied in categorising whether the specific expiratory breath is categorised as incomplete, or complete.

In yet an embodiment, the step of c) comparing first input to a corresponding reference level comprises comparing a measure of the average of incomplete expiratory breaths to a corresponding reference level for the measure average of incomplete expiratory breaths associated with at risk of having dynamic hyperinflation and/or increased autoPEEP.

The second input may also be further specified. Thus, in an embodiment, the second input indicative of the carbon dioxide level in expired gas of the subject is applied for estimating the end expiratory carbon dioxide level for a specific breath (EtCO2). The skilled person would understand that various ways of finding EtCO2 are already available.

In yet an embodiment, the end expiratory carbon dioxide level for a specific breath (EtCO2) is however applied for a number of breaths to estimate a measure for the variability of the end expiratory carbon dioxide level of said subject, which is particularly advantageous and not previously seen. The term ‘variability’ may be synonymously and/or equivalently used with terms such as dispersion, scatter, and/or spread, as the person skilled in statistical analysis will readily understand, these measures essentially how small or extended a given distribution, or sample, is. Variability may for example be quantified by measures such as the variance, the standard deviation, the interquartile range etc.

In yet a further embodiment, the step of c) comparing second input to a corresponding reference level comprises comparing the measure for the variability of the end expiratory carbon dioxide level to a corresponding reference level for the variability of the end expiratory carbon dioxide level associated with at risk of having dynamic hyperinflation and/or increased autoPEEP.

The method according to the invention may be used to define further categories of associated risks. Thus, in an embodiment the step of c) comparing whether first and second input deviate significantly from said reference levels is additionally applied for categorising said subject into three, or more, categories of risks having dynamic hyperinflation and/or increased autoPEEP. In e.g. FIG. 4 and corresponding text, an example with three categories is described. It is to be understood that categories may be divided based on e.g. the degree of deviation from a threshold level or e.g. based on several threshold levels dividing the different categories.

In a similar embodiment, the step of c) comparing whether first and second input deviate significantly from said corresponding inputs provided from said subject from a previous point in time is additionally applied for categorising changes in the risk of the subject having dynamic hyperinflation and/or increased autoPEEP.

The different input may be supplied from different devices. Thus, in an embodiment, the first and/or the second input is provided from a mechanical ventilator supporting the subject, preferably said subject has spontaneous breathing effort. In a similar embodiment, the second data is provided from a capnometer, e.g. connected to a mechanical ventilator. Capnometer allow measurement of the partial pressure of carbon dioxide in the respiratory gas.

In yet a detailed embodiment, said subject
requires mechanical ventilation; and/or
has spontaneous breathing effort; and/or
is not fully sedated.

The different input may be averaged over a number of breaths. Thus, in another embodiment, the first input and/or second input is averaged over a number of breaths, such as in the range 5-50 breaths, such as 5-30 breaths, such as 10-25 breaths. The first input may then be processed to a number of incomplete breaths is then given as a number (below the maximum range) or a fraction value or frequency value. The second input may be given as a partial pressure (typically in the range of approximately 0-6 kPa but some times approximately 0-15 kPa) but in the present context, the second input is preferably processed to give a fraction of expiratory carbon dioxide (FCO2) in percentage or similar, the fraction is often approximately 0-5% depending on the medical condition, cf. FIG. 4. In FIGS. 2-5 and corresponding text, a rolling window of 20 breaths has been applied.

In a specific embodiment, the first input indicative of the expiratory flow of the patient is the flow.

In another specific embodiment, the second input is the end tidal carbon dioxide. This end-tidal value is that calculated. In yet an embodiment, the input signal is the respiratory gas CO2 level (either the fraction, partial pressure, or mass), which is further calculated to provide the end tidal carbon dioxide.

In a fifth aspect the invention relates to a mechanical ventilation system, preferably for partly supported ventilation, the system being arranged for determining the risk of having dynamic hyperinflation and/or increased auto positive end expiratory pressure (autoPEEP) in a subject (P), the system comprising

ventilation means capable of mechanical ventilating said patient with air and/or one or more medical gases,
control means, the ventilator means being controllable by said control means by operational connection thereto,
measurement means (M1, M2) arranged for measuring a first input indicative of the expiratory flow of the subject and the second input indicative of the carbon dioxide level in expired gas of the, the measurement means being capable of delivering
a first input (D1), indicative of the expiratory flow of the subject; and
a second input (D2), indicative of the carbon dioxide level in expired gas of the subject; and
   the control means being arranged for comparing said first and second input to one or more corresponding reference levels (REF1, REF2); and if said first and second input deviate significantly from said one or more reference levels, said subject is at risk of having dynamic hyperinflation and/or increased autoPEEP; or if said first and second input do not deviate significantly from said one or more reference levels, said subject is not at risk of having dynamic hyperinflation and/or increased autoPEEP.

In a sixth aspect the invention relates to a computer program product being adapted to enable a computer system comprising at least one computer having data storage means in connection therewith to control a mechanical ventilation system according to the fifth aspect, or to control and implement the method of the first, the second, the third and/or the fourth aspect.

This aspect of the invention is particularly, but not exclusively, advantageous in that the present invention may be accomplished by a computer program product enabling a computer system to carry out the operations of the mechanical ventilation system, respectively, when down- or uploaded into the computer system. Such a computer program product may be provided on any kind of computer readable medium, or through a network.

The individual aspects of the present invention may each be combined with any of the other aspects. These and other aspects of the invention will be apparent from the following description with reference to the described embodiments.

### BRIEF DESCRIPTION OF THE FIGURES

The invention will now be described in more detail with regard to the accompanying figures. The figures show one way of implementing the present invention and is not to be construed as being limiting to other possible embodiments falling within the scope of the attached claim set.

FIG. 1 is a flow chart of the method according to the present invention.

FIG. 2 illustrates analysis of example data describing expiratory flow. A) Expiratory flow over time. B) The flow profile of a series of breaths from the same patient. Complete breaths are labelled with stars (*). C) Average number of incomplete breaths over a rolling window of 20 breaths for the data illustrated in FIG. 2B.

FIG. 3 illustrates analysis of example data describing end expiratory carbon dioxide levels. A) A typical CO2 profile on expiration. The highest value of CO2 on this profile represents the end-expired values. B) Variability of expiratory carbon dioxide in a series of breaths from the same patient. C) Two times the standard deviation of the end-tidal CO2 level over a rolling window of 20 breaths, illustrating the variability in end-tidal CO2.

FIG. 4 illustrates three classifications of dynamic hyperinflation based upon averages of incomplete breaths and end-expiratory CO2 variability. A) The two curves on the left hand side illustrate flow and CO2 profiles over a number of breaths for the same patient without dynamic hyperinflation. The figure on the right hand side shows the patterns of the average number of incomplete breaths (solid line) and the pattern of CO2 variability (dotted line) in the same patient. B) The two curves on the left hand side illustrate flow and CO2 profiles over a number of breaths for the same patient with moderate dynamic hyperinflation. The figure on the right hand side shows the patterns of the average number of incomplete breaths (solid line) and the pattern of CO2 variability (dotted line) in the same patient. C) The two curves on the left hand side illustrate flow and CO2 profiles over a number of breaths for the same patient with severe dynamic hyperinflation. The figure on the right hand side shows the patterns of the average number of incomplete breaths (solid line) and the pattern of CO2 variability (dotted line) in the same patient.

FIG. 5 illustrates identification of change in the classification state of dynamic hyperinflation from moderate to none. A) Breath by breath CO2 profile. B) Flow profile over the breaths from A). Complete expiration is marked with stars. C) Averages of the number of incomplete breaths (solid line) and CO2 variability (dotted line). The vertical line illustrates the beginning of change in respiratory pattern of the patient.

FIG. 6 shows a mechanical ventilation system (100) according to the present invention, where a patient (P) is supported, fully or partly, in the ventilation and the mechanical ventilation system comprising measurement means for the flow, and the measurement means for carbon dioxide in the expired gas of the subject.

### DETAILED DESCRIPTION OF AN EMBODIMENT

This invention is a method/computer system for identifying the degree and changes in the nature of dynamic hyperinflation. Key to this invention is the application of signals describing expiratory flow and/or expiratory carbon dioxide level, and analysis of trends of changes in variables derived from these signals.

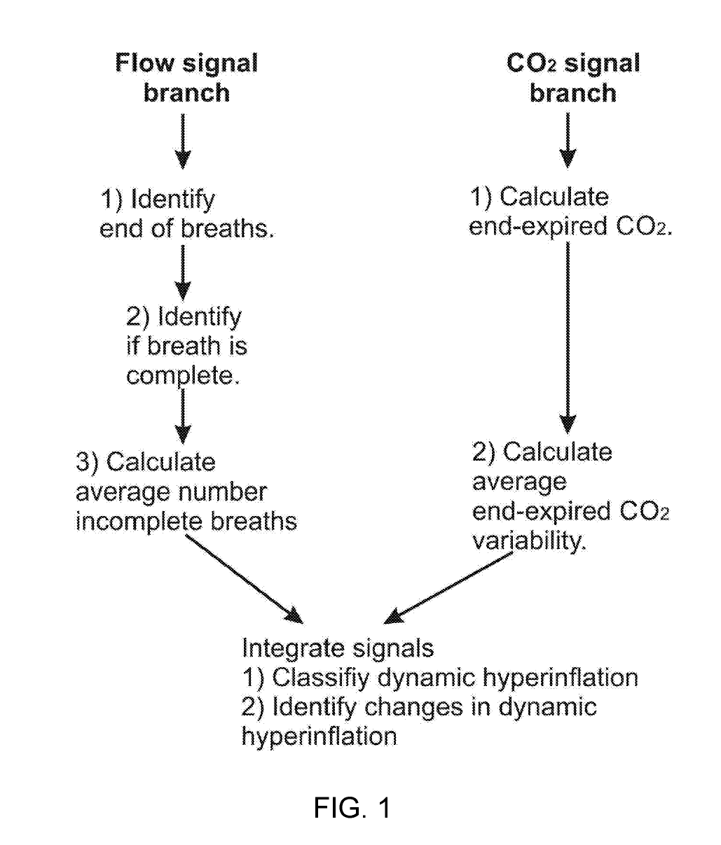

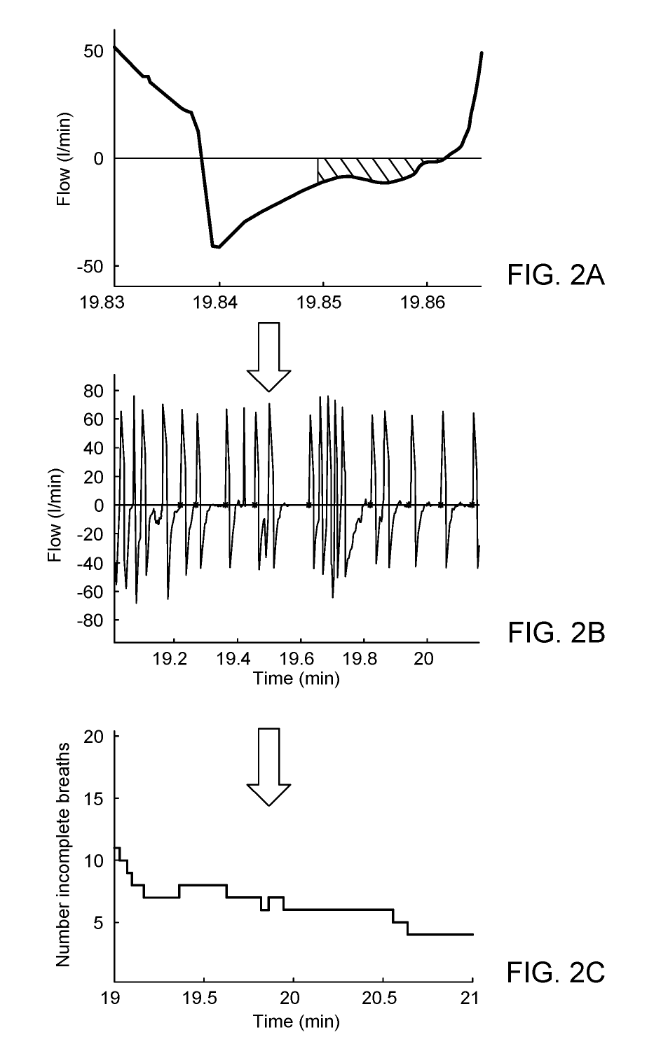

FIG. 1 is a schematic drawing of a specific embodiment of the method of the invention. The method, illustrated on the figure, includes two arms for analysing the expiratory flow (flow signal branch) and carbon dioxide signal (carbon signal branch), respectively, and their subsequent combination. Steps 1 and 2 of the flow signal analysis (FIG. 1) are illustrated in FIG. 2. For each breath the end of the breath can be automatically detected, this can be done by a number of methods including analysis of the positive or negative nature of the flow signal over a period of time. Following detection of the breath end, the flow signal a fixed time interval prior to this end can be integrated to give a volume over this period (FIG. 2A). This fixed time interval can be determined either as a value, or as a percentage of the expiratory time. If this volume is greater that a threshold value then the breath is considered incomplete, otherwise it is considered complete. The threshold for determining an incomplete breath can be either a fixed value, or a percentage of the expiratory volume. FIG. 2B illustrates a series of breaths from the same patient. Complete breaths are labelled with stars (*) plotted at the end of the breath. For patients with spontaneous breathing effort, breathing can be highly heterogeneous. This is illustrated in FIG. 2B where only some of the breaths are complete. The third step in describing the flow signal (FIG. 1, step 3) is therefore to calculate an average. FIG. 2C illustrates the average number of incomplete breaths over a rolling window. In the FIG. 2C), the rolling window is set to 20 breaths, meaning that the y axis represents the number of incomplete breaths ranging from 0 to 20.

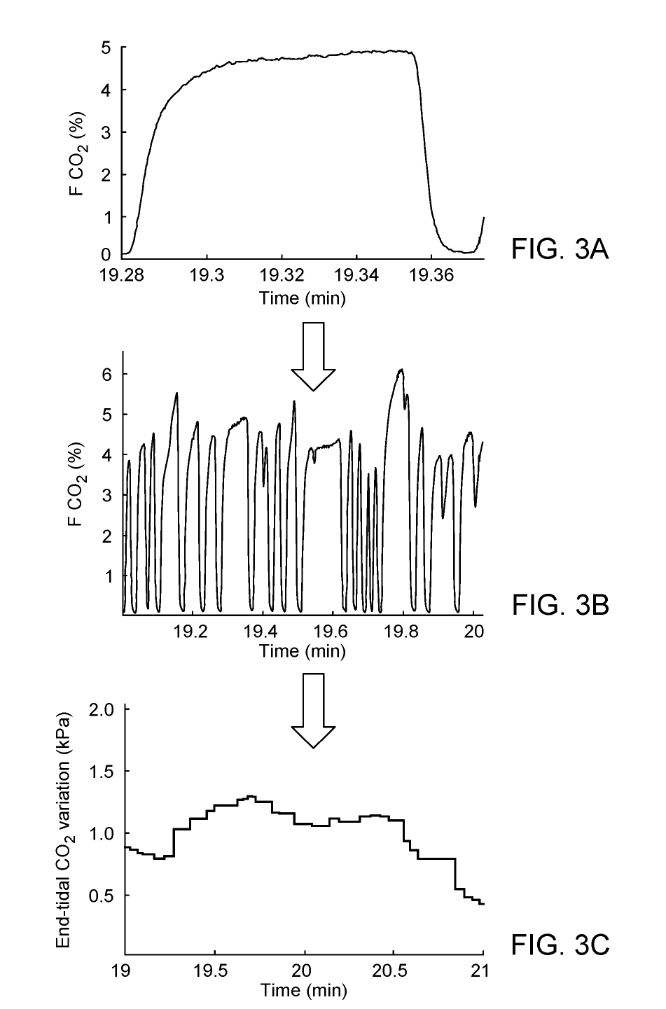

Steps 1 and 2 of the carbon dioxide (CO2) signal analysis (FIG. 1) are illustrated in FIG. 3. A typical CO2 profile on expiration is shown in FIG. 3A. The highest value of CO2 on this profile represents the end-expired value. Typically, this is at the end of the plateau where gas is expired from the smallest units of the lung, i.e. the alveoli. This plateau is usually seen on a complete expiration. For incomplete expirations the plateau may not be reached and end-tidal values might therefore be lower. For a breathing pattern with heterogeneity, i.e. some complete and some incomplete breaths, it is likely that one will see some breaths reaching plateau and some not, meaning that end tidal values will have a great deal of variability. Those not reaching plateau may indicate that the patient is at increased risk of dynamic hyperinflation. This variability is illustrated by a number of breaths in FIG. 3B. This variability can be calculated by considering the average value of end expired CO2 over a rolling window of breaths and the standard deviation of the variability in end expired CO2. The standard deviation of the CO2 signal reflects the variability in the end expired CO2 and hence the heterogeneity of the breathing pattern. FIG. 3C illustrates a plot of 2 times the standard deviation over a rolling window of 20 breaths, i.e. step 2 of the CO2 signal analysis branch.

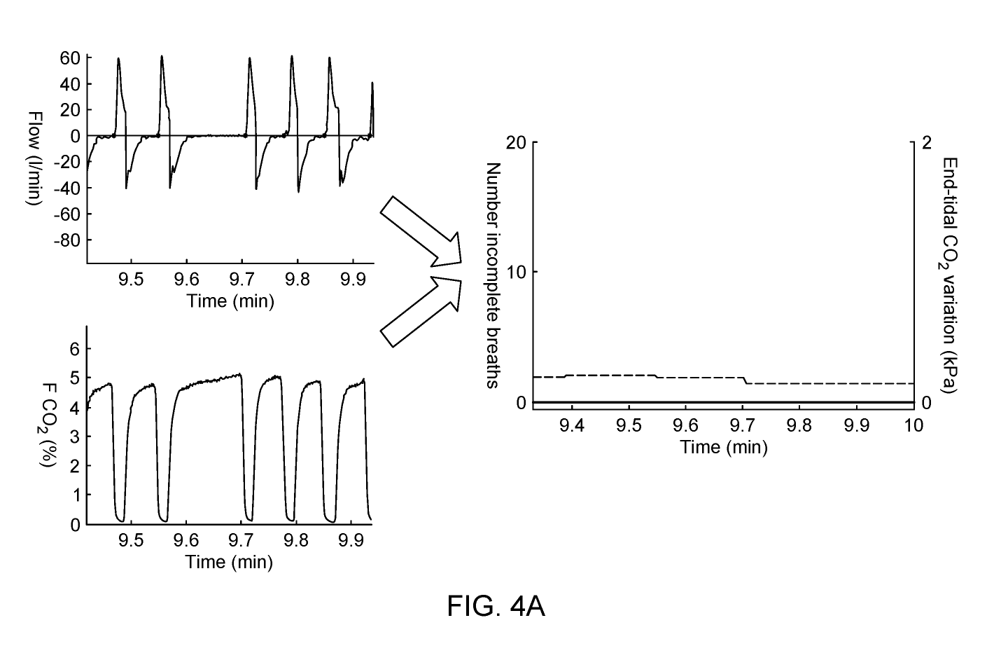

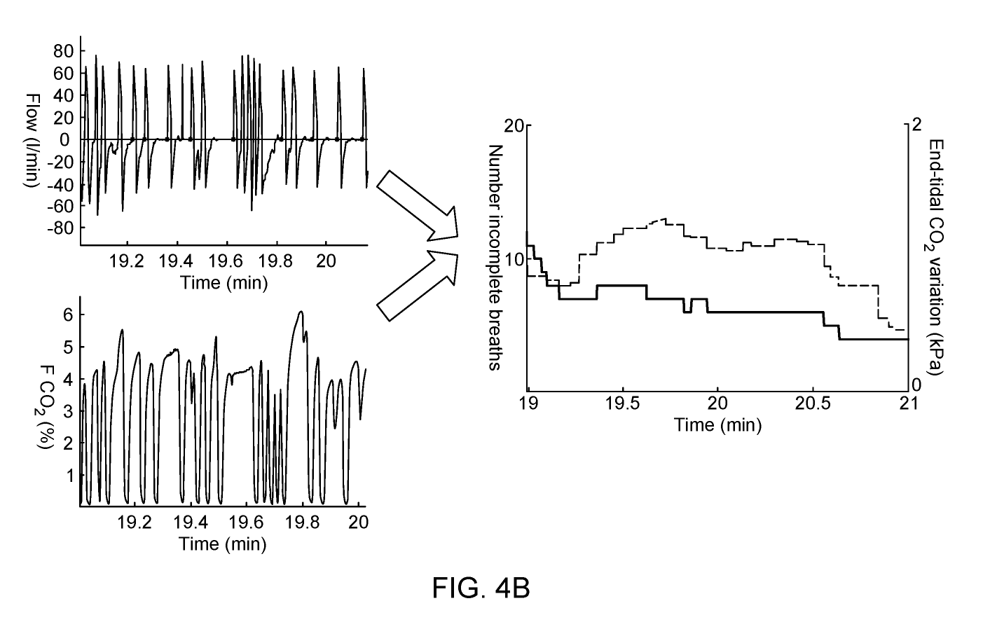

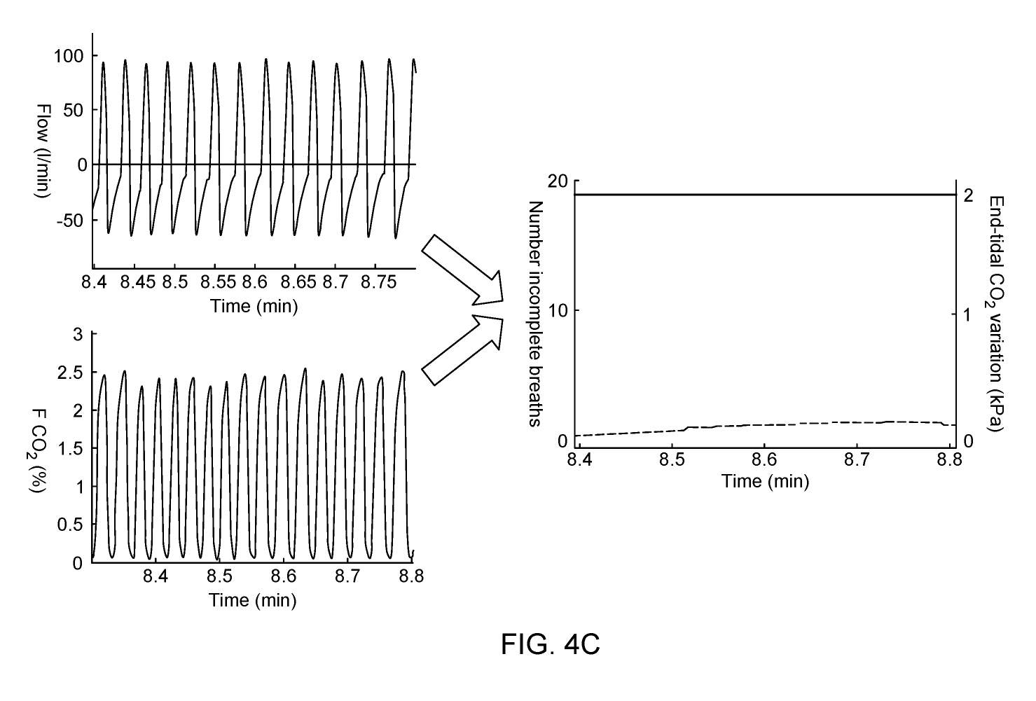

The fact that the average number of incomplete breaths and the average variability in the end tidal CO2 signal both provide information concerning the degree of dynamic hyperinflation means that these signals can be considered together and used to evaluate the status of the patient, i.e. step 5 of the method illustrated in FIG. 1. In particular, the method can classify the degree of dynamic hyperinflation into 3 or more categories. FIG. 4 illustrates examples of 3 such categories. FIG. 4A illustrates flow and CO2 profiles (left hand side) and the patterns of the average number of incomplete breaths (solid line) and the pattern of CO2 variability (dotted line) (right hand side) in a patient without dynamic hyperinflation. The number of incomplete breaths approximates zero, and the CO2 variability is very low. FIG. 4B illustrates flow and CO2 profiles (left hand side) and the patterns of the average number of incomplete breaths (solid line) and CO2 variability (dotted line) (right hand side) in patients with moderate dynamic hyperinflation. The number of incomplete breaths ranges from 5-11 over the 20 breath window, and the CO2 variability is high. The high CO2 variability suggesting that the breaths are heterogeneous, with some reaching completion and a high expired CO2 level, while others ending prematurely and reaching a low expired CO2 level. FIG. 4C illustrates flow and CO2 profiles (left hand side) and patterns of the average number of incomplete breaths (solid line) and of CO2 variability (dotted line) (right hand side) in a patient with extreme dynamic hyperinflation. The number of incomplete breaths is around 19 per 20 breaths, and the CO2 variability is very low. This pattern suggests that the breaths are homogeneous, with all expirations ending prematurely as indicated by the lack of plateau in the CO2 signals, and as such resulting in little variation in expired CO2 values.

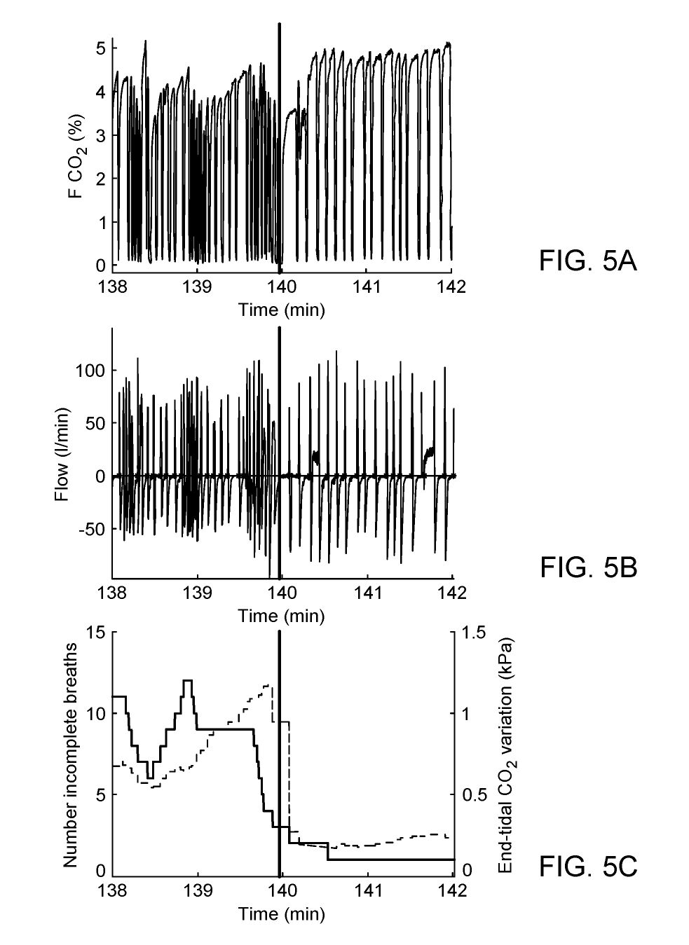

Analysis of the number of incomplete breaths and expired CO2 variability, as described in this method, can also be used to identify changes in the patient's state with regard to dynamic hyperinflation. FIG. 5 illustrates the situation where the patient goes from a moderate degree of hyperventilation to little or none. FIG. 5A illustrates the breath by breath CO2 profile, FIG. 5B illustrates the flow profile over these breaths with complete expiration marked with stars, and FIG. 5C the averages of the number of incomplete breaths (solid line) and CO2 variability (dotted line). The vertical line illustrates the beginning of change in respiratory pattern of the patient, end tidal CO2 variability decreases, the number of incomplete breaths decreases. The average values of these, seen in FIG. 5C, shown clearly the movement of the patient from a picture similar to that of FIG. 4B i.e. moderate dynamic hyperinflation, to one similar to that of 4A i.e. little or no dynamic hyperinflation.

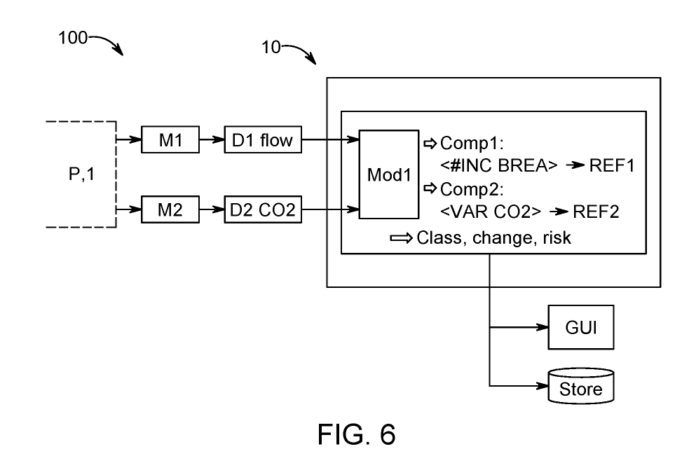

FIG. 6 shows a mechanical ventilation system 100 according to the present invention, preferably for partly supported ventilation. The system is particularly arranged for determining the risk of a subject P, 1 of having dynamic hyperinflation and/or increased auto positive end expiratory pressure (autoPEEP).

The system comprises ventilation means capable of mechanical ventilating said patient with air and/or one or more medical gases (not shown), and control means 10, the ventilator means being controllable by said control means by operational connection thereto.

Also measurement means M1 and M2 are arranged for measuring a first input indicative of the expiratory flow of the subject, e.g. a flow measuring device typically present in a mechanical ventilation system, and the second input indicative of the carbon dioxide level in expired gas of the, e.g. a capnometers, which allow measurement of the partial pressure of carbon dioxide in the respiratory gas.

The measurement means are particularly capable of delivering
a first input D1, indicative of the expiratory flow of the subject, indicated as ‘FLOW’;
   and
a second input D2, indicative of the carbon dioxide level in expired gas of the subject, indicated as ‘CO2’; and

Additionally, the control means 10 are arranged for comparing said first and second input to one or more corresponding reference levels REF1, REF2; and if said first and second input deviate significantly from said one or more reference levels, said subject is at risk of having dynamic hyperinflation and/or increased autoPEEP; or if said first and second input do not deviate significantly from said one or more reference levels, said subject is not at risk of having dynamic hyperinflation and/or increased autoPEEP. In one particular advantageous embodiment, the first input D1 is processed to give an average number of incompletely breaths, shown as <# INC BREA>, in a suitable rolling time window, e.g. 20 breaths, and the second input D2 is processed to give an average variability of end tidal carbon dioxiode, shown as <VAR CO2>, cf. FIGS. 4 and 5 and corresponding description above.

The control means 10 may particularly have installed a computer program product being adapted to enable a computer system comprising at least one computer having data storage means in connection therewith to control a mechanical ventilation system as shown in FIG. 6. Suitable user interfaces, e.g. graphical user-interface GUI may be provided, along with appropriate storage STORE for later use and/or continuous monitoring of the patient P.

Glossary

| | |
|---|---|
| ICU | Intensive Care Unit |
| EtCO2 | End tidal CO2 |
| AutoPEEP | Auto Positive End Expiratory Pressure |

### REFERENCES

1. The Acute Respiratory Distress Syndrome Network. Ventilation with Lower Tidal Volumes as Compared with Traditional Tidal Volumes for Acute Lung Injury and the Acute Respiratory Distress Syndrome. N Engl J Med 2000; 342:1301-1308.
2. Marini J M. Dynamic Hyperinflationand auto-positive end -exipiratory pressure: Lessons learned over 30 years. American Journal of Respiratory and Critical Care Medicine. 2011; 184:756-62.
3. Laghi F, Goyal A. Auto-PEEP in respiratory failure. Minerva Anestesiologica. 2012; 78:201-21.
4. Blanch L, Bernabé F, Lucangelo U. Measurement of air trapping, intrinsic positive end-exiratory pressure, and dynamic inflation in mechanically ventilated patients. Respiratory Care 2005; 50, 110-124.
5. Nguyen Q T, Pastor D, Lellouche F, L'Her E. Mechanical ventilation systems monitoring: automatic detection of dynamic hyperinflation and asynchrony. 35^{th} Annual International Conference of the IEEE EMBS. 5207-5210.
6. Nguyen Q T, Pastor D, L′Her E. automatic detection of AutoPEEP during controlled mechanical ventilation. Biomedical Engineering Online 2012; 11:32.
7. Lourens M S, van den Berg B, Aerts J G J V, Verbraak A F M, Hoogsteden H C, Bogaard J M. Expiratory time constants in mechanically ventilated patients with and without COPD. Intensive Care Medicine 2000; 26:1612-1618.

All of the above patent and non-patent literature are hereby incorporated by reference in their entirety.

1. A computer-implemented method for determining the risk of a subject of having dynamic hyperinflation and/or increased auto positive end expiratory pressure (autoPEEP) in a subject, the method comprising:
a) providing a first input, indicative of the expiratory flow of the subject; and
b) providing a second input, indicative of the carbon dioxide level in expired gas of the subject; and
c) comparing said first and second input to one or more corresponding reference levels; and
d) if said first and second input deviate significantly from said one or more reference levels, said subject is at risk of having dynamic hyperinflation and/or increased autoPEEP; or if said first and second input do not deviate significantly from said one or more reference levels, said subject is not at risk of having dynamic hyperinflation and/or increased autoPEEP.

2. A computer-implemented method for determining a change in dynamic hyperinflation and/or auto positive end expiratory pressure (autoPEEP) in a subject, the method comprising
a) providing a first input indicative of the expiratory flow profile of the subject; and
b) providing a second input indicative of the carbon dioxide level in expired gas of the subject; and
c) comparing said first and second input to corresponding input provided from said subject from a previous point in time; and
d) if said first and second input deviate significantly from said one or more corresponding input provided from said subject from a previous point in time, said subject is at risk of having dynamic hyperinflation and/or increased autoPEEP; or if said first and second input do not deviate significantly from said one or more corresponding input provided from said subject from a previous point in time, said subject is not at risk of having dynamic hyperinflation and/or increased autoPEEP.

3. The method according to claim 2, wherein the first input indicative of the expiratory flow of the subject is further integrated over time for an individual expiratory breath of the subject, and if the integrated area is below a certain threshold area then the specific expiratory breath is categorised as incomplete, and/or if the integrated area is above a certain threshold area then the specific expiratory breath is categorised as complete.

4. The method according to claim 3, wherein a value of the time derivative of the first input at the end of the said specific expiratory breath is additionally applied in categorising whether the specific expiratory breath is categorised as incomplete, or complete.

5. The method according to claim 3, wherein the step of c) comparing first input to a corresponding reference level comprises comparing a measure of the average of incomplete expiratory breaths to a corresponding reference level for the measure average of incomplete expiratory breaths associated with at risk of having dynamic hyperinflation and/or increased autoPEEP.

6. The method according to claim 1, wherein the second input indicative of the carbon dioxide level in expired gas of the subject is applied for estimating the end expiratory carbon dioxide level for a specific breath (EtCO2).

7. The method according to claim 6, wherein the end expiratory carbon dioxide level for a specific breath (EtCO2) is applied for a number of breaths to estimate a measure for the variability of the end expiratory carbon dioxide level of said subject.

8. The method according to claim 7, wherein the step of c) comparing second input to a corresponding reference level comprises comparing the measure for the variability of the end expiratory carbon dioxide level to a corresponding reference level for the variability of the end expiratory carbon dioxide level associated with at risk of having dynamic hyperinflation and/or increased autoPEEP.

9. The method according to claim 8, wherein the step of c) comparing whether first and second input deviate significantly from said reference levels is additionally applied for categorising said subject into three, or more, categories of risks having dynamic hyperinflation and/or increased autoPEEP.

10. The method according to claim 8, wherein the step of c) comparing whether first and second input deviate significantly from said corresponding inputs provided from said subject from a previous point in time is
additionally applied for categorising changes in the risk of the subject having dynamic hyperinflation and/or increased autoPEEP.

11. The method according to claim 2, wherein the first and/or the second input is provided from a mechanical ventilator supporting the subject, and said subject has spontaneous breathing effort.

12. The method according to claim 2, wherein the first input and/or second input is averaged over a number of breaths, such as in the range 5-50, such as 5-30, such as 10-25.

13. The method according to claim 2, wherein the first input indicative of the expiratory flow of the patient is the flow.

14. The method according to claim 2, wherein the second input is the end tidal carbon dioxide.

15. A mechanical ventilation system, for partly supported ventilation, the system being arranged for determining the risk of having dynamic hyperinflation and/or increased auto positive end expiratory pressure (autoPEEP) in a subject (P), the system comprising:
a ventilator capable of mechanical ventilating said patient with air and/or one or more medical gases,
a control, the ventilator means being controllable by said control means by operational connection thereto,
measurement means arranged for measuring a first input indicative of a expiratory flow of the subject and the second input indicative of a carbon dioxide level in expired gas of the, the measurement means being capable of delivering
a first input, indicative of the expiratory flow of the subject; and
a second input, indicative of the carbon dioxide level in expired gas of the subject; and
the control being arranged for comparing said first and second input to one or more corresponding reference levels; and if said first and second input deviate significantly from said one or more reference levels, said subject is at risk of having dynamic hyperinflation and/or increased autoPEEP; or if said first and second input do not deviate significantly from said one or more reference levels, said subject is not at risk of having dynamic hyperinflation and/or increased autoPEEP.

16. A computer program product being adapted to enable a computer system (10) comprising at least one computer having data storage means in connection therewith to control a mechanical ventilation system according to claim 16, or to control and implement the method of claim 2.
